(19) United States
(12) Patent Application Publication
(10) Pub. No.: US 2022/0142545 A1
(43) Pub. Date: May 12, 2022

(54) HIGH DENSITY ELECTRODE CATHETERS

(71) Applicant: St. Jude Medical, Cardiology Division, Inc., St. Paul, MN (US)

(72) Inventor: Gregory K. Olson, Elk River, MN (US)

(21) Appl. No.: 17/437,337

(22) PCT Filed: Mar. 5, 2020
(86) PCT No.: PCT/US20/21158

§ 371 Date: Sep. 8, 2021

Related U.S. Application Data
(60) Provisional application No. 62/815,652, filed on Mar. 8, 2019.

(51) Int. Cl. A61B 5/287; A61B 5/367; A61B 5/263; A61B 5/00

(52) U.S. Cl. CPC A61B 5/287; A61B 5/6852; A61B 5/263; A61B 5/367

(57) ABSTRACT

An electrophysiology system for mapping tissue includes a catheter having a plurality of electrodes. The system may be a catheter having a dense collection of small electrodes in fixed positions on its tip. The system may be an electrophysiology apparatus having a catheter, the catheter having a body with a proximal end and a distal end. At the distal end of the catheter body is a distal tip comprising a plurality of electrodes and/or coaxtrodes. A signal processor may be operably connected to the plurality of electrodes and/or coaxtrodes and can measure at least one electrophysiological parameter.

### CROSS-REFERENCE TO RELATED APPLICATIONS

This application claims the benefit of U.S. provisional application No. 62/815,652, filed 8 Mar. 2019, which is hereby incorporated by reference as though fully set forth herein.

### BACKGROUND

The present disclosure relates generally to catheters having a plurality of electrodes for use inside the human heart during medical procedures. In particular, the instant disclosure relates to catheters having a dense collection of small electrodes on the tip of the catheter. The electrodes can be used to derive parameters such as transmembrane current, local conduction velocity, and tissue impedance. The catheters can be used in electrophysiological mapping, such as may be performed in cardiac diagnostic and therapeutic procedures.

The heart contains two specialized types of cardiac muscle cells. The majority, around ninety-nine percent, of the cardiac muscle cells are contractile cells, which are responsible for the mechanical work of pumping the heart. The second type of cardiac muscle cells are autorhythmic cells, which function as part of the autonomic nervous system to initiate and conduct action potentials responsible for the contraction of the contractile cells. The cardiac muscle displays a pacemaker activity, in which membranes of cardiac muscle cells slowly depolarize between action potentials until a threshold is reached, at which time the membranes fire or produce an action potential. This contrasts with a nerve or skeletal muscle cell, which displays a membrane that remains at a constant resting potential unless stimulated. The action potentials, initiated by the autorhythmic cardiac muscle cells, spread throughout the heart triggering rhythmic beating without any nervous stimulation.

An arrhythmia occurs when the cardiac rhythm becomes irregular, i.e., too fast (tachycardia) or too slow (bradycardia), or the frequency of the atrial and ventricular beats are different. Arrhythmias can develop from either altered impulse formation or altered impulse conduction. Arrhythmias can be either benign or more serious in nature depending on the hemodynamic consequences of arrhythmias and their potential for changing into lethal arrhythmias.

Electrophysiological mapping, and more particularly electrocardiographic mapping, is a part of numerous cardiac diagnostic and therapeutic procedures, such as procedures to treat the foregoing arrhythmias. Typically, such electrophysiology studies employ electrophysiology devices, such as catheters, that include one or more electrodes capable of measuring the electrical activity occurring on the epicardial or endocardial surface, or at other locations on or near the heart. The resultant data set can be used to generate a map of the cardiac electrical activity, which the practitioner can then utilize to develop a course of action (e.g., to identify locations for ablation). For example, electrode traces, e.g., intracardiac electrogram traces, can be stacked vertically on a display, with the order of the traces corresponding to the order of electrodes on the electrophysiology catheter.

### BRIEF SUMMARY

The current disclosure provides solutions to problems with deriving electrophysiological parameters such as transmembrane current, local conduction velocity, and tissue impedance. In general, disclosed herein are catheter systems having a dense collection of small electrodes to measure a large number of surface potentials within a small area. For example, the catheter systems may be used to acquire and analyze electrograms. In addition, the quantity and distribution of the electrodes allows for some not to be in contact with tissue, which facilitates the non-contact electrodes to be compared with others more likely in contact. The comparison of the contact and non-contact electrodes can be used for comparing and contrasting impedance and near/far field signals for signal to noise improvement.

One embodiment is a catheter for use in an electrophysiology procedure such as electrocardiographic mapping, the apparatus including a catheter and a signal processor. The apparatus may be packaged as part of a kit.

The catheter includes an elongated catheter body having a proximal end and a distal end. A handle is operably connected to the proximal end of the body. Positioned at the distal end is an atraumatic distal tip. In some embodiments, the width of the distal tip is greater than the width of the distal end of the catheter body.

The distal tip may include a nonconductive material. Located on the outer surface of the atraumatic distal tip are a plurality of electrodes. A first region of the outer surface has a first subset of the plurality of electrodes, and a second region of the outer surface has a second subset of the plurality of electrodes. The first region and the second region have the same surface areas. The first subset of the plurality of electrodes includes a greater number of electrodes than the second subset of the plurality of electrodes. In one embodiment, the first subset of the plurality of electrodes are uniformly distributed through the first region, and the second subset of the plurality of electrodes are uniformly distributed throughout the second region. In some embodiments, the distal tip has a conductive material, and the electrodes are electrically insulated from the conductive material. In some embodiments, within each subset of the plurality of electrodes, the interelectrode spacing between each of the electrodes may be between about 0.1 mm to about 0.5 mm edge to edge.

The electrodes may be microelectrodes, ring electrodes, and/or dot electrodes (also known as circle or spot electrodes). In some embodiments, the electrodes are spot electrodes surrounded by ring electrodes, referred to herein as “coaxtrodes.” In other embodiments, the distal tip contains a combination of coaxtrodes and spot electrodes. In other embodiments, the distal tip contains only microelectrodes, and the microelectrodes are all the same size. In yet other embodiments, the distal tip contains only coaxtrodes.

The signal processor is operably connected to the plurality of electrodes to receive and analyze electrical signals in order to derive at least one electrophysiological parameter. For example, the electrophysiological parameter may be transmembrane current, tissue impedance, local conduction velocity, and any combinations thereof.

Another embodiment is an apparatus for use in an electrophysiology procedure. The apparatus includes a catheter and a signal processor. The catheter has a body with a proximal end and a distal tip region. Contained on the distal tip region are a plurality of electrodes. The signal processor is operably connected to the plurality of electrodes and is able to measure at least one electrophysiological parameter. In some embodiments, the plurality of electrodes are biased toward one side of the distal tip region. The plurality of electrodes may be spaced equally from each other. In some embodiments, the electrodes are microelectrodes, and the microelectrodes are all the same size. In other embodiments the electrodes are coaxtrodes. In some embodiments, the distal tip has a conductive material, and the electrodes are electrically insulated from the conductive material. In yet other embodiments, the distal tip region includes a nonconductive material. Each of the electrodes may be spaced between about 0.1 mm to about 0.5 mm edge to edge.

Another embodiment is a catheter that includes an elongate catheter body that has a proximal end and a distal end. A handle is operably connected to the proximal end. A distal tip is connected to the distal end. The width of the distal tip may be greater than the width of the distal end of the elongate catheter body. Contained on the distal tip is an array of electrodes, the electrodes being distributed uniformly. In some embodiments, the array of electrodes is biased toward one side of the distal tip. The electrodes may be microelectrodes that are all the same size. In other embodiments, the electrodes may be coaxtrodes or a combination of microelectrodes and coaxtrodes. In some embodiments, the microelectrodes are spaced between about 0.1 mm to about 0.5 mm edge to edge.

Another embodiment is a catheter that includes an elongated catheter body. The elongated catheter body includes a proximal end and a distal end. Located at the distal end is a distal tip. Contained on the outer surface of the distal tip is a plurality of electrodes. A first region of the outer surface of the distal tip includes a first subset of the plurality of electrodes, and a second region of the outer surface of the distal tip includes a second subset of the plurality of electrodes. The first region and the second region have the same surface areas. The first subset of the plurality of electrodes may include a greater number of electrodes than the second subset of the plurality of electrodes. In some embodiments, the first subset of the plurality of electrodes includes a first section of electrodes and a second section of electrodes, and the density of electrodes in the first section is different from the density of electrodes in the second section.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 depicts an exemplary distal portion of a catheter that can be used in connection with aspects of the instant disclosure.

FIG. 3 depicts an exemplary distal portion of a catheter that can be used in connection with aspects of the instant disclosure.

FIG. 4 is a close-up plan view of the top of the distal tip of the catheter depicted in FIG. 2, wherein the electrodes are microelectrodes.

FIG. 5 is a close-up plan view of the top of the distal tip of the catheter depicted in FIG. 2, wherein the electrodes are coaxtrodes.

FIGS. 6A through 6D depict various side profiles of the distal tip of the catheter depicted in FIG. 2. FIG. 6A depicts the right side profile, FIG. 6B depicts the front side profile, FIG. 6C depicts the back side profile, and FIG. 6D depicts the left side profile.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The present disclosure provides methods, apparatuses, and systems for the creation of electrophysiology maps (e.g., electrocardiographic maps). For purposes of illustration, several exemplary embodiments will be described in detail herein in the context of a cardiac electrophysiology procedure. It is contemplated, however, that the methods, apparatuses, and systems described herein can be utilized in other contexts.

For purposes of illustration, aspects of the disclosure will be described in detail herein in the context of a cardiac mapping procedure carried out using an electrophysiology mapping system (e.g., using an electroanatomical mapping system such as the EnSite Precision™ cardiac mapping system from Abbott Laboratories of Abbott Park, Ill.).

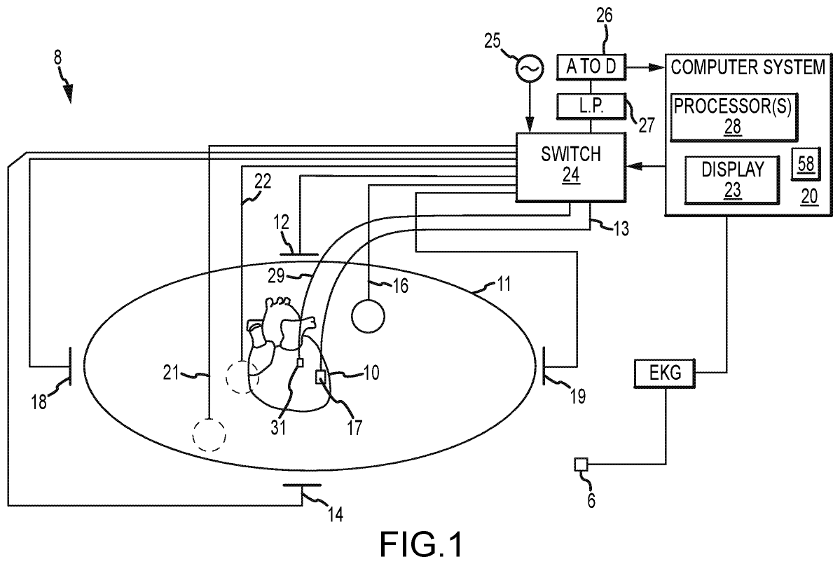

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example, to create a diagnostic data map of the patient's heart 10.

As one of ordinary skill in the art will recognize, and as will be further described below, system 8 determines the location, and in some aspects the orientation, of objects, typically within a three-dimensional space, and expresses those locations as position information determined relative to at least one reference.

For simplicity of illustration, the patient 11 is depicted schematically as an oval. In the embodiment shown in FIG. 1, three sets of surface electrodes (e.g., patch electrodes) are shown applied to a surface of the patient 11, defining three generally orthogonal axes, referred to herein as an x-axis, a y-axis, and a z-axis. In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 16, 22 are applied along a third axis generally orthogonal to both the x-axis and the y-axis, such as along the sternum and spine of the patient in the thorax region, and may be referred to as the Chest and Back electrodes. The heart 10 lies between these pairs of surface electrodes 12/14, 18/19, and 16/22.

An additional surface reference electrode (e.g., a “belly patch”) 21 provides a reference and/or ground electrode for the system 8. The belly patch electrode 21 may be an alternative to a fixed intra-cardiac electrode 31, described in further detail below. It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple (e.g., eight) splines, each of which in turn includes multiple (e.g., eight) electrodes.

The foregoing embodiments are merely exemplary, however, and any number of electrodes and/or catheters may be used. For example, in some embodiments, a high density mapping catheter, such as the Ensite™ Array™ non-contact mapping catheter of Abbott Laboratories, can be utilized.

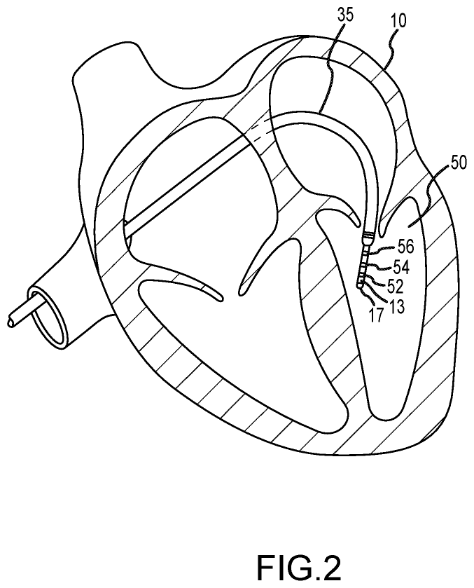

Likewise, it should be understood that catheter 13 (or multiple such catheters) are typically introduced into the heart and/or vasculature of the patient via one or more introducers and using familiar procedures. For purposes of this disclosure, a segment of an exemplary catheter 13 is shown in FIG. 2. In FIG. 2, catheter 13 extends into the left ventricle 50 of the patient's heart 10 through a transseptal sheath 35. The use of a transseptal approach to the left ventricle (e.g., across the intra-atrial septum and through the mitral valve) is well known and will be familiar to those of ordinary skill in the art, and need not be further described herein. Of course, catheter 13 can also be introduced into the heart in any other suitable manner, and may also be introduced into any chamber of the heart consistent with application of the teachings herein.

Catheter 13 includes electrode 17 on its distal tip, as well as a plurality of additional measurement electrodes 52, 54, 56 spaced along its length in the illustrated embodiment. Typically, the spacing between adjacent electrodes will be known, though it should be understood that the electrodes may not be evenly spaced along catheter 13 or of equal size to each other. Since each of these electrodes 17, 52, 54, 56 lies within the patient, location data may be collected simultaneously for each of the electrodes by system 8.

Similarly, each of electrodes 17, 52, 54, and 56 can be used to gather electrophysiological data from the cardiac surface (e.g., surface electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate a graphical representation of a cardiac geometry and/or of cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., surface electrode pairs 12/14, 18/19, and 16/22) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are exposed to the field from a current pulse and are measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data sets from each of the surface electrodes, the internal electrodes, and the virtual electrodes may all be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In some embodiments, system 8 is the EnSite™ Velocity™ or EnSite Precision™ cardiac mapping and visualization system of Abbott Laboratories. Other localization systems, however, may be used in connection with the present teachings, including for example the RHYTHMIA HDX™ mapping system of Boston Scientific Corporation (Marlborough, Mass.), the CARTO navigation and location system of Biosense Webster, Inc. (Irvine, Calif.), the AURORA® system of Northern Digital Inc. (Waterloo, Ontario), Sterotaxis' NIOBE® Magnetic Navigation System (Stereotaxis, Inc., St. Louis, Mo.), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to graphically representing multiple electrophysiological characteristics on a single surface model. Accordingly, system 8 can also include a modeling module 58. Modeling module 58 can be used, inter alia, to graphically represent two or more electrophysiological characteristics (e.g., two or more electrophysiology maps) on a single geometric model (e.g., a single cardiac geometry).

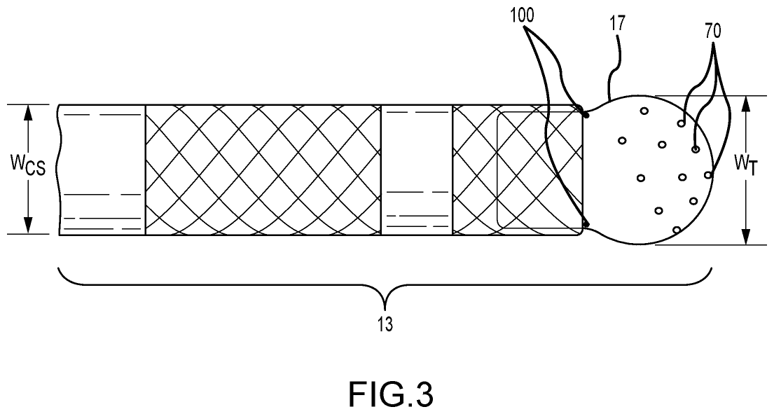

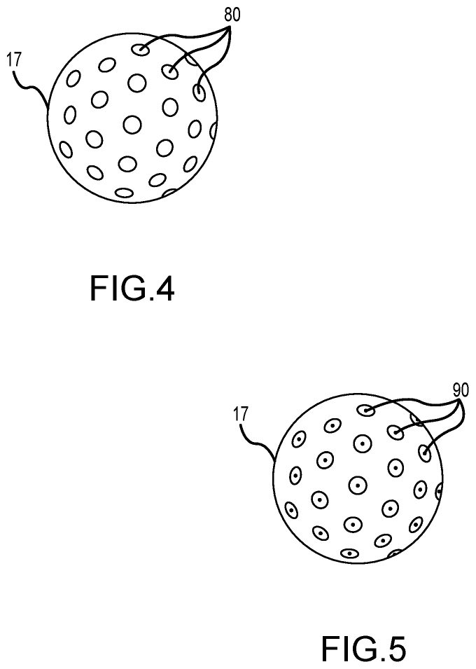

As described above, embodiments of the disclosure relate to a catheter 13 that includes a plurality of electrodes thereon. FIG. 3 depicts a representative catheter 13 including a plurality of electrodes 70 on its distal tip 17. Electrodes 70 may be microelectrodes 80, as shown in FIG. 4, coaxtrodes 90, as shown in FIG. 5, or a combination of microelectrodes 80 and coaxtrodes 90. As used herein, the term “coaxtrode” refers to a spot electrode surrounded by a ring electrode. Coaxtrodes are described in U.S. Patent Application No. 61/919,176, which is hereby incorporated by reference in its entirety. Advantageously, coaxtrodes are capable of providing a direction independent measure of activation time. In other words, by using coaxial electrodes, signal analysis is independent of the direction of signal propagation or tip orientation. For example, the coaxtrodes 90 can enable determination of surface potentials within a small area following larger-scale mapping and/or signal propagation direction independent of device orientation. The coaxtrode configuration is able to provide an orientation-independent bipolar electrogram. This is in contrast to conventional ring electrode pairs that produce bipolar electrograms that are dependent on the orientation relative to the depolarization wavefront. Distal tip 17 may also include contact-sensing electrodes 100.

As shown in FIG. 3, the width of the distal tip 17 (W_{T}) is greater than the width of the catheter shaft (W_{CS}). In some embodiments, the width of the distal tip 17 W_{T} is at least 1%, 5%, 10%, 15%, 20%, or 25% wider than the width of the catheter shaft W_{CS}. In other embodiments, if a more compact catheter is required, the width of the distal tip 17 W_{T} is the same as the width of the catheter shaft W_{CS}. The catheter shaft 85 may be from about 4-10 French, more preferably from about 4-8 French.

The electrodes 70 may have a diameter of about 0.010 mm to about 0.5 mm. In some embodiments, the electrodes 70 have a diameter of 0.010 mm to about 0.25 mm. In other embodiments, the electrodes 70 have a diameter of 0.010 mm.

The distance between electrodes 70 can be measured from the center of a first electrode to the center of a second electrode (“c/c” for center to center). The ratio for the spacing of electrodes 70 c/c to the size of the diameter of the electrodes can be from about 0.25:1 to about 4:1. In some embodiments the ratio for the spacing of electrodes 70 c/c to the size of the diameter of the electrodes is 2.5:1. In other embodiments, the ratio for the spacing of electrodes 70 c/c to the size of the diameter of the electrodes is 1:1.

The electrodes 70 may be located on the distal tip 17 in a uniform manner. The electrodes 70 may all have the same diameters. In a preferred embodiment, the electrodes 70 have a uniform size.

The electrodes 70 could also be configured to extract propagation direction by vector loop mapping. Epicardial potential differences are often described as a vector representation. Depending on the amount of divergence among vector angles near the maximum amplitude, loops have been classified as narrow, open or hooked. In the normal myocardium, open loops are thought to be caused by a change of direction of propagation, and hooked loops by discontinuous conduction. The distal tip as shown in FIG. 3 can be used to analyze superficial extracellular potentials during depolarization.

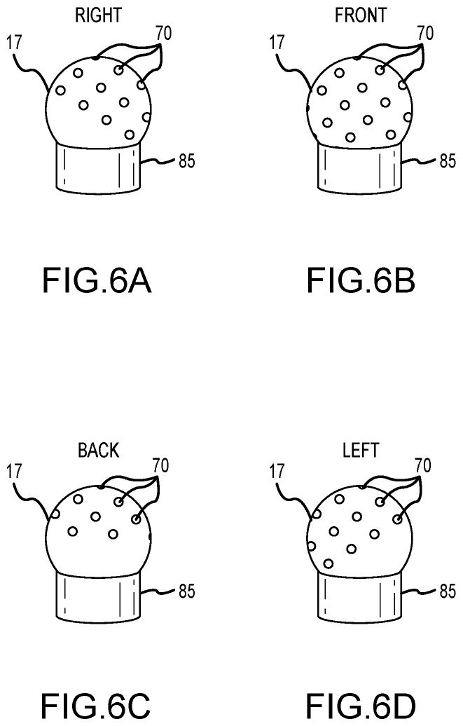

As shown in FIGS. 6A-6D, the electrodes 70 are uniformly spaced on the distal tip 17, with the electrodes 70 biased toward one side of the distal tip 17. The orientation of the electrodes 70 on the distal tip 17 allow doctors to get catheters into small locations.

The distal tip 17 may comprise a conductive material. In another embodiment, the distal tip 17 comprises a nonconductive material. In this embodiment, each of the electrodes 70 are electrically insulated.

An amplifier is operably coupled to electrodes 70 in order to amplify the signals received by electrodes 70. In some embodiments, the amplifier is located within distal tip 17, for example in a position proximal to the electrodes. Alternatively, the amplifier can be located within a handle at the proximal end of catheter 13, or even external to catheter 13.

The amplifier can be multiplexed to all of the electrodes 70. In other aspects, a unique amplifier is operably coupled to each electrode. Indeed, both one-to-one and one-to-many correspondence between amplifiers and electrodes 70 are contemplated.

Conductors from the electrodes 70 to amplifier(s) may be configured as a twisted pair, coaxial, triaxial or shielded twisted pair in order to mitigate noise. In the case of triaxial or shielded twisted pair, the outermost shield could be tied to a ring conductor or other large surface area electrode submerged in a blood pool.

Catheter 13 (or multiple such catheters) are typically introduced into the heart and/or vasculature of the patient via one or more introducers and using familiar procedures. Indeed, various approaches to introduce catheter 13 into a patient's heart, such as transseptal approaches, will be familiar to those of ordinary skill in the art, and therefore need not be further described herein.

Since each electrode 70 lies within the patient, location data may be collected simultaneously for each electrode 70 by system 8. Similarly, each electrode 70 can be used to gather electrophysiological data from the cardiac surface (e.g., surface electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate a graphical representation of a cardiac geometry and/or of cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

The measured voltages may be used by system 8 to determine the location in three-dimensional space of the electrodes inside the heart, such as roving electrodes 70 relative to a reference location, such as reference electrode 31. That is, the voltages measured at reference electrode 31 may be used to define the origin of a coordinate system, while the voltages measured at roving electrodes 70 may be used to express the location of roving electrodes 70 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A catheter comprising:
an elongated catheter body comprising a proximal end and a distal end; and
an atraumatic distal tip at the distal end of the catheter body, wherein the distal tip comprises an outer surface;
wherein:
the outer surface of the distal tip comprises a plurality of electrodes;
a first region of the outer surface comprises a first subset of the plurality of electrodes and a second region of the outer surface comprises a second subset of the plurality of electrodes;
the first region and second region have the same surface areas; and
the first subset of the plurality of electrodes includes a greater number of electrodes than the second subset of the plurality of electrodes.

2. The catheter of claim 1, wherein the first subset of the plurality of electrodes are uniformly distributed throughout the first region and the second subset of the plurality of electrodes are uniformly distributed throughout the second region.

3. The catheter of claim 2, wherein the plurality of electrodes comprises a plurality of microelectrodes, and wherein the plurality of microelectrodes are all the same size.

4. The catheter of claim 2, wherein the plurality of electrodes comprises a plurality of coaxtrodes.

5. The catheter of claim 1, wherein the width of the distal tip is greater than the width of the distal end of the catheter body.

6. The catheter of claim 3, wherein the distal tip comprises a nonconductive material.

7. The catheter of claim 3, wherein the distal tip comprises a conductive material, and wherein the plurality of microelectrodes are electrically insulated from the conductive material of the distal tip.

8. The catheter of claim 2, wherein an interelectrode spacing between the electrodes in the plurality of electrodes is between 0.1 mm to 0.5 mm edge to edge.

9. An apparatus for use in an electrophysiology procedure, comprising:
a catheter comprising a body having a proximal end and a distal tip region;
a plurality of electrodes positioned within the distal tip region, wherein the plurality of electrodes are biased toward one side of the distal tip region; and
a signal processor operably connected to the plurality of electrodes, wherein the signal processor measures at least one electrophysiological parameter.

10. The apparatus of claim 9, wherein each of the electrodes in the plurality of electrodes are spaced equally from each other.

11. The apparatus of claim 10, wherein the electrodes are microelectrodes, and wherein the microelectrodes are all the same size.

12. The apparatus of claim 10, wherein the electrodes are coaxtrodes.

13. The apparatus of claim 9, wherein the distal tip region comprises a nonconductive material.

14. The apparatus of claim 11, wherein the distal tip region comprises a conductive material, and wherein each of the microelectrodes are individually electrically insulated.

15. The apparatus of claim 10, wherein the electrodes in the plurality of electrodes are spaced between 0.1 mm to 0.5 mm edge to edge.

16. A catheter comprising:
an elongate catheter body having a proximal end and a distal end;
a handle operably coupled to the proximal end of the elongate catheter body; and
a distal tip connected to the distal end of the elongate catheter body, wherein the distal tip comprises an array of electrodes comprising a uniform distribution of electrodes, and wherein the array of electrodes is biased to one side of the distal tip.

17. The catheter of claim 16, wherein, the width of the distal tip is greater than the width of the distal end of the elongate catheter body.

18. The catheter of claim 16, wherein the electrodes are microelectrodes, and wherein the microelectrodes are all the same size.

19. The catheter of claim 16, wherein the electrodes are coaxtrodes.

20. The catheter of claim 18, wherein the microelectrodes are spaced between 0.1 mm to 0.5 mm edge to edge.

21. A catheter comprising:
an elongated catheter body comprising a proximal end and a distal end; and
a distal tip at the distal end of the catheter body, wherein the distal tip comprises an outer surface;
wherein:
the outer surface of the distal tip comprises a plurality of electrodes;
a first region of the outer surface comprises a first subset of the plurality of electrodes and a second region of the outer surface comprises a second subset of the plurality of electrodes;
the first region and second region have the same surface areas;
the first subset of the plurality of electrodes includes a greater number of electrodes than the second subset of the plurality of electrodes;
the first subset of the plurality of electrodes comprises a first section of electrodes and a second section of electrodes; and
the density of electrodes differs between the first section and second section of electrodes.
